(19) United States
(12) Patent Application Publication
(10) Pub. No.: US 2010/0041124 A1
(43) Pub. Date: Feb. 18, 2010

(54) MULTI-STAGE CSTR BIOREACTOR SYSTEM EQUIPPED WITH CELL RECYCLE UNIT

(75) Inventors: Ho Nam Chang, Daejeon (KR); Byoungjin Kim, Seoul (KR); Changmoon Jeong, Daejeon (KR); Jong Won Kang, Seoul (KR); Joongkon Park, Daegu (KR)

Correspondence Address: MOORE & VAN ALLEN PLLC, P.O. BOX 13706, Research Triangle Park, NC, 27709, US

(73) Assignee: KOREA ADVANCED INSTITUTE OF SCIENCE AND TECHNOLOGY, Daejeon (KR)

(21) Appl. No.: 12/523,279

(22) PCT Filed: Jan. 24, 2008
(86) PCT No.: PCT/KR2008/000432

§ 371 Date: Oct. 20, 2009

(30) Foreign Application Priority Data
Jan. 25, 2007 (KR) 10-2007-0008052

(51) Int. Cl. C12N 1/00; C12M 1/14

(52) U.S. Cl. 435/243; 435/299.1

(57) ABSTRACT

The present invention relates to a single CSTR bioreactor equipped with a cell recycle unit, and a multi-stage CSTR bioreactor system in which 2-7 such single CSTR bioreactors are connected in series with each other. According to the present invention, multi-stage fermentation is carried out in the bioreactor equipped with the cell recycle unit, and thus the production of products (alcohols, organic acids, antibiotics, recombinant proteins, etc.) by anaerobic or aerobic microbial fermentation can be increased, and in addition, a final product can be obtained at high concentration, thus improving the productivity and economic efficiency of a microbial process.

### TECHNICAL FIELD

The present invention relates to a single CSTR bioreactor equipped with a cell recycle unit, and a multi-stage CSTR bioreactor system in which 2-7 such single CSTR bioreactors are connected in series with each other.

### BACKGROUND ART

Microorganisms produce various substances useful to humans in a metabolic process. Various substances, including alcohols which are normally consumed by humans, monosodium L-glutamate (MSG), penicillin, various enzymes for use as cleaners, amino acids such as lysine, lactic acid, acetic acid, human growth hormones caused by gene recombination, and insulin, are produced through microbial fermentation.

Generally, these substances are mostly produced in a batch or fed-batch operation. In this operation, a process comprising filling a reactor with media, sterilizing the medium, inoculating the media with bacteria, culturing the bacteria for a given period and purifying the cultured bacteria is carried out. This process is characterized in that a substrate such as glucose is consumed to the maximum extent so as to increase the yield of a product, thus maximizing the final concentration of the product. In other words, this process aims to maximize the conversion rate of a substrate, the yield and final concentration of a product.

However, the batch or fed-batch culture does not consider a problem of how much a product can be produced in a reactor having a specified size within a unit time.

That is, because the batch or fed-batch culture comprises the several-step process as described above, it is inefficient, and thus has low productivity.

In an attempt to overcome this problem, a chemical process, in which substrate is continuously introduced into a catalyst-packed bed so as to increase the efficiency and productivity of the process, has also recently been adopted. However, if a method of immobilizing microorganisms is used, the clogging of the packed bed can occur due to the excessive expression by microorganisms.

For this reason, most fermentation processes employ a method of producing products through suspension culture rather than immobilization. However, where continuous fermentation is carried out through this suspension culture, there is a limitation in increasing the productivity of the process, due to wash out of cells.

Meanwhile, the proliferation rate (dX/dt) of cells in unit volume of a reactor can be expressed as the following equation:

dX/dt=D(X₁X₀)+(μ−kd)X [1]
wherein D is dilution rate, X is cell concentration, X₀ is the concentration of cells introduced into the reactor, X₁ is the concentration of cells discharged from the reactor, μ is the production rate of cells, and kd is the death rate of cells.

Herein, (μ−kd)X should be larger than D(X1−X₀) in order to prevent wash out of cells. However, the productivity of the process is in proportion to DX, and thus if the operation is carried out at low D in order to prevent the washing out of cells, the productivity of the process will be decreased. For this reason, in attempts to increase the productivity, various methods of collecting cells from a flow discharged from a reactor have been designed.

Methods of collecting cells include a precipitation method and a method which uses hollow fibers. Currently, the two methods are all used in the treatment of municipal sewage, but are not used in biological processes due to low permeation rate and the phenomenon that microorganisms adhere to the surface of a membrane. In order to increase the productivity of the microbial process, a continuous operation should be carried out while increasing “DX” of the above equation [1], but continuous high-concentration culture has not been realized due tor the above-described reasons (Shuler, M. L. and Kargi, F., Bioprocess Engineering, Basic Concepts, Prentice Hall, 2002).

Recently, an apparatus and method of culturing cells by allowing a reaction medium to flow through a biocompatible, macroporous ceramic particle-packed bed disposed in a cell culture reactor were disclosed. In the prior art, the production rate of cellular waste products was significantly reduced by solving the problem that the medium must be recycled in order to attain better medium utilization and cost effectiveness. However, there is a shortcoming in that it is not easy to obtain the macroporous ceramic particles as packing materials (U.S. Pat. No. 5,262,320).

Also, a method, which comprises obtaining a packing material in an economic manner compared to the prior reactor comprising a packed bed, packing the porous bioreactor with a support material in order to increase the recovery rate of cells, and then culturing cells using a radial flow of a reaction medium, was developed (U.S. Pat. No. 4,833,083). The cell culture method has an advantage in that it is applicable to large-scale applications in the commercial production of biologically and/or therapeutically important molecules using cells, cell cultures, cell components, enzymes, enzyme systems and the like, but the efficiency of the cell culture process has not yet been improved.

In addition, there have been various attempts associated with cell culture, including a process of promoting the contact between a packed bed of contact particles and a liquid-gas feed in a reactor vessel (U.S. Pat. No. 4,559,132), a method for the in vitro culture of animal cells to recover cell-secreted proteins (U.S. Pat. No. 5,102,790), a process and system for culturing adhesive cells in a packed bed of solid cell matrix (U.S. Pat. No. 5,260,211), and a method of culturing cells on microporous beads located in a fixed bed, containing anionic polysaccharide gel or polyelectrolyte membrane (KR 1991-0006475).

Meanwhile, continuous high-concentration culture operation, which is a cell culture method having high productivity, has a shortcoming in that utility and process stability are low because of low product concentration compared to the batch or fed-batch culture (Lee, C. W. and Chang, H. N., Biotechnol Bioeng, 29:1105, 1987).

Accordingly, in the art to which the present invention pertains, in order to solve the above-described problems, there is an urgent need to develop cell culture technology, which can increase productivity and, at the same time, the concentration of a product.

Thus, the present inventors have made extensive efforts to solve the above-described problems occurring in the prior art and, as a result, have found that, when a high concentration of a product is obtained by operating a multi-stage CSTR system, in which a packed bed as a cell recycle unit is included in a bioreactor, and five bioreactors are connected in series, the culture of microorganisms can be carried out in a more economic manner, thereby completing the present invention.

### SUMMARY OF THE INVENTION

It is a main object of the present invention to provide single CSTR and multi-stage CSTR bioreactor systems equipped with a cell recycle unit.

To achieve the above object, the present invention provides a single CSTR bioreactor system in which a cell recycle unit serving to block the discharge of cells from the bioreactor and, at the same time, discharge culture broth except for cells during a culture process, is included in the bioreactor.

In another aspect, the present invention provides a multi-stage CSTR reactor system in which 2-7 such single CSTR bioreactor systems are connected in series with each other.

In still another aspect, the present invention provides a method for culturing microorganisms, the method comprises using said single CSTR or multi-stage CSTR bioreactor system.

Other features and embodiments of the present invention will be further apparent from the following detailed description and the attached claims.

### BRIEF DESCRIPTION OF DRAWINGS

FIG. 1 shows a multi-stage bioreactor system equipped with an upflow packed bed as a cell recycle unit.

FIG. 2 shows the results of continuous high-concentration culture of yeast, conducted using the multi-stage bioreactor system equipped with the upflow packed bed as the cell recycle unit.

FIG. 3 shows the combination of various types of cell recycle units with a bioreactor (a: a bioreactor equipped with a fully packed bed; b: a bioreactor equipped with a partially packed bed; c: a bioreactor equipped with a cylinder packed bed, a duct packed bed or a plate packed bed; and d: a bioreactor equipped with a multi-bed).

FIG. 4 shows the results of computer modeling, which show the concentrations of a single antibody, obtained in a 1-7-step continuous culture process using a CSTR bioreactor equipped with a cylindrical depth filter as a cell recycle unit.

### DETAILED DESCRIPTION OF THE INVENTION, AND PREFERRED EMBODIMENTS

As used herein, the term “cell recycle unit” means a unit which serves to block the discharge of cells from a bioreactor and discharge cultured broth except for cells. The use of such a cell recycle unit enables the high-concentration culture of cells to be achieved.

As used herein, the term “multi-stage” means that pluralities of bioreactors are connected. For example, two-stage means that two bioreactors are connected with each other, and three-stage means that three bioreactors are connected.

As used herein, the term “high-purity oxygen” means O₂ having a purity of more than 99%.

In one aspect, the present invention relates to a single CSTR bioreactor system in which a cell recycle unit serving to block the discharge of cells from the bioreactor and, at the same time, discharge culture broth except for cells during a culture process, is equipped.

In the present invention, the cell recycle unit is preferably selected from the group consisting of a packed bed, a cylindrical depth filter and a membrane cell recycle unit.

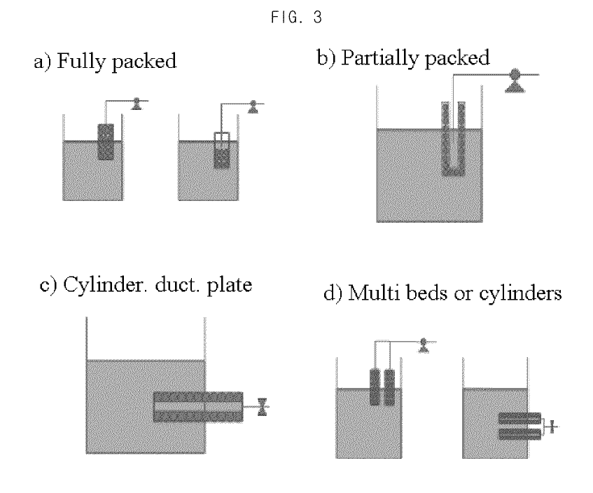

In the present invention, examples of the packed bed may be a fully packed bed, a partially packed bed, a cylinder or pipe packed bed, a duct packed bed, a plate packed bed and the like, as shown in FIG. 3. Alternatively, the packed bed may also be a multi-packed bed consisting of a plurality of said packed beds connected with each other. The packed bed is packed with a packing material, selected from the group consisting of activated carbon, ceramic particles and polymer particles, to a porosity of 28-70%.

In the present invention, the cylindrical depth filter as the cell recycle unit preferably consists of a filter having a pore size gradually decreasing from the outside to the inside thereof (Lee, J. C. et al., Biotech. Progress., 21:134-139, 2005).

In the present invention, the membrane cell recycle unit may be in the form of hollow fiber described in Lee, C. W. and Chang, H. N., Biotech. Bioeng., 29:1105-1112, 1987. However, any membrane cell recycle unit may be used without limitation in the present invention, as long as it can be included in the bioreactor to isolate cells from a culture broth.

In the present invention, the cells are preferably selected from the group consisting of bacteria, yeasts, molds, animal cells and plant cells.

The bioreactor that is used in the present invention preferably additionally comprises fundamental bioreactor components, which are conventionally used in the art, including a pH-adjusting unit, a temperature control unit, a substrate supply unit, a DO control unit.

In another aspect, the present invention relates to a multi-stage CSTR reactor system in which 2-7 such single CSTR bioreactor systems are connected in series with each other.

In the CSTR bioreactor according to the present invention, productivity can be increased by culturing cell products at high concentration using the cell recycle unit such as a packed bed through a multi-step process, compared to the prior precipitation method or the method of recycling cells into a porous fine tube, which have been frequently used in microbial processes.

The prior packed bed has been frequently used to adsorb materials or remove fine particles. That is, when fluid containing adsorbent materials or fine particles is allowed to flow toward the lower portion of the packed bed, the adsorbent materials or the fine particles will be adsorbed or entrapped on particles packed in the packed bed to be removed. The removal process is carried out through precipitation, surface rejection and depth filtration, and when the packed bed reaches a saturated state to increase the pressure such that the fluid does not flow through the packed bed, the packed bed will be regenerated using a backwashing method and used again.

In the multi-stage CSTR bioreactor of the present invention, the cell recycle unit performs a function similar to a membrane. A general membrane has properties of blocking the passage of cells through the surface thereof and allows only culture broth to pass therethrough. Herein, the general membrane blocks 100% of cells. However, the cell recycle unit according to the present invention blocks 90-99% of cells, and thus the high-concentration culture of a cell product can be achieved in a more efficient manner by introducing some of cells in a bioreactor into the next bioreactor and further culturing the introduced cells.

Meanwhile, in the multi-stage CSTR bioreactor system according to the present invention, the cell recycle units equipped in the respective bioreactors may have slightly different functions.

In the multi-stage CSTR bioreactor system according to the present invention, the cell recycle unit included in the final bioreactor is preferably constructed such that it discharges only cultured broth except for cells, and the cell recycle units included in the remaining bioreactors are preferably constructed such that they recycle most of cells in the bioreactors without discharging the cells from the bioreactors and, at the same time, introduce some of the cells into the next bioreactor.

For example, in a 7-stage CSTR bioreactor system, the first to sixth cell recycle units included in the first to sixth bioreactors, respectively, are preferably constructed such that they can recycle cells during culture, and then deliver a cultured products containing some of cells, into the next-stage bioreactor, whereas the seventh cell recycle unit included in the seventh bioreactor, which is the final bioreactor, is preferably constructed such that it discharges a cultured broth except for cells.

That is, the first to sixth cell recycle units are preferably packed to a porosity of 28-70%, and the seventh cell recycle unit is preferably packed such that the discharge of cells is further blocked. In an alternative embodiment, the cell recycle unit included in the final bioreactor of the multi-stage CSTR bioreactor system (the seventh cell recycle unit in this case) may also be a membrane-type cell recycle unit.

In still another aspect, the present invention relates to a method for culturing microorganisms, the method comprises using said single CSTR or multi-stage CSTR bioreactor system.

In the present invention, said microorganisms are preferably selected from the group consisting of anaerobic, facultative and aerobic microorganisms.

In the case where facultative or aerobic microorganisms are cultured using the single CSTR or multi-stage CSTR bioreactor system according to the present invention, it is preferable to mount a high-purity oxygen regeneration unit in the single CSTR or multi-stage CSTR bioreactor system and supply oxygen into the bioreactor through the oxygen regeneration unit.

In the culture method according to the present invention, cell culture and/or the yield of product can be enhanced through the additional supply of a substrate into the bioreactor or the control of temperature, and the efficiency of microorganism isolation can be increased by rotating or backwashing the cell recycle units included in the system.

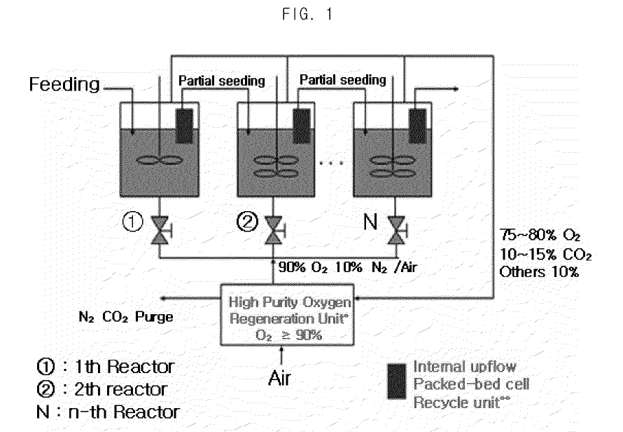

According to a preferred embodiment of the present invention, a three-stage CSTR bioreactor system, in which a first bioreactor, a second bioreactor and a third bioreactor are continuously connected in series, can be constructed (FIG. 1).

The first and second bioreactors of the three-stage CSTR bioreactor system are equipped with first and second cylindrical upflow packed beds as cell recycle units, respectively. The first and second upflow packed beds may be packed with activated carbon to a porosity of 33%. Also, the third upflow packed bed is preferably packed with activated carbon with a porosity different from the porosities of the first and second packed beds, such that it can block the discharge of cells from the third bioreactor and discharge only a cultured product. Moreover, a cell recycle unit based on a membrane capable of completely blocking the discharge of cells may also be used.

In the case where aerobic microorganisms are cultured, a high-purity oxygen regeneration unit for supplying oxygen is connected to the lower portion of each of the first bioreactor, the second bioreactor and the third bioreactor.

Then, the culture of aerobic microorganisms can be carried out using the three-stage CSTR bioreactor system in the following manner.

When aerobic culture of microorganisms inoculated into the first bioreactor is carried out, most of the cultured microorganisms remain in the first bioreactor by the first upflow packed bed, and some of the cells are introduced into the second bioreactor through the first upflow packed bed. At this time, the remaining substrate and product, which are not consumed in the first bioreactor, are also simultaneously introduced into the second bioreactor, and if necessary, a fresh substrate can be introduced into the second bioreactor to further increase productivity. The recycle of cells in the second bioreactor and the introduction of a cultured broth into the third bioreactor are carried out in the same manner as described above. Herein, oxygen required for the anaerobic culture of microorganisms in the bioreactor system is supplied by the high-purity oxygen regeneration unit.

Although the three-stage CSTR bioreactor system has been illustrated in the above embodiment, it will be apparent to those skilled in the art that, depending on the number of bioreactors, two-stage or 4-7-stage CSTR bioreactor systems can be embodied.

### Examples

Hereinafter, the present invention will be described in further detail with reference to examples. It will be apparent to one skilled in the art that these examples are for illustrative purpose only and are not construed to limit the scope of the present invention.

### Example 1

### Cell Recycle Experiment in Single CSTR Bioreactor System Equipped with Upflow Packed Bed

A cell recycle culture in a bioreactor system equipped with an upflow packed bed was carried out using Saccharomyces cerevisiae (ATCC 24858), as a bacterial strain, and 100 g/L of a glucose solution, as a substrate. Herein, a reactor having a volume of 2 L was equipped with a packed bed, in which activated carbon (0.8-2 mm diameter) was packed in a glass tube, having a length of 480 mm and an inner diameter of 13 mm, to a porosity of 32%. The reactor system was operated at a temperature of 30° C., a pH of 6.5 and a dilution rate (D) of 0.1/h.

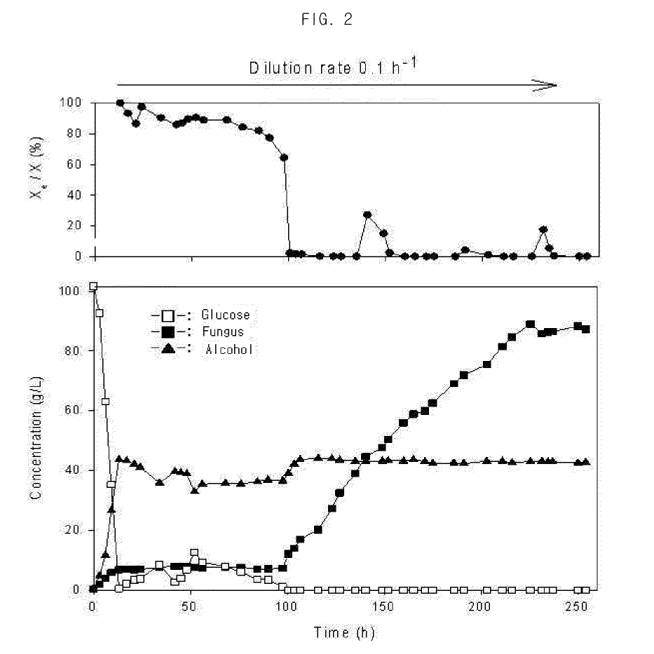

As a result, as shown in FIG. 2, glucose was almost consumed to 10-20 g/L within about 10 hours from 100 g/L at the initial reaction stage, and was completely consumed to 0 g/L at about 100 hours after the start of the operation.

Also, the concentration of cells in the reactor was maintained at 10 g/L up to 100 hours, and then started to increase to 80 g/L. The concentration of alcohol was maintained at about 40 g/L during a period ranging from 10 hours to 250 hours. The relative concentration (Xe/X %) of cells discharged out of the reactor was maintained at 60-100% up to 90 hours, and then decreased to almost 0% after about 100 hours, suggesting that the packed bed functioned as a membrane, and thus little or no cells were discharged from the reactor. However, because the cells would be continuously accumulated in the packed bed, the cells were discharged from the reactor in the form of small peaks at about 140 hours, 200 hours and 230 hours after the start of the operation. As a result, it could be confirmed that the packed bed successfully functioned like a membrane 100 hours after the start of the operation.

Meanwhile, although only the packed bed was illustrated as the cell recycle unit in this Example, the use of a cell recycle unit in the form of (a) a fully packed bed, (b) a partially packed bed, (c) a cylinder packed bed, a duct packed bed, a plate packed bed, or (d) a multi packed bed, as shown in FIG.3, is also encompassed within the scope of the present invention.

### Example 2

### Removal Efficiency of Microorganisms According to Porosity of Activated Carbon in Packed Bed

When the porosity of activated carbon in Example 1 was reduced to less than 28%, 100% of the microorganisms were removed, and when the porosity was more than 70%, microorganisms were not substantially removed. Also, when the porosity was less than 28%, the packed bed was completely filled with microorganisms, thus the operation of the packed bed became impossible. Consequently, it could be seen that, when the porosity of the packed bed was 28-70%, the removal efficiency of microorganisms was the highest.

### Example 3

### Removal Efficiency of Microorganisms According to Packing Material

It was observed that the removal rate of microorganisms was 0.5% in the case where stone was used as the packing material for the packed bed in the same conditions as in Example 1, 3% in the case where finely cut porous microfiber was used, 6% in the case where a ceramic material was used, and 94% in the case where activated carbon was used. As a result, it could be seen that the removal rate of microorganisms was most excellent when activated carbon was used as the packing material for the packed bed.

### Example 4

### Relationship Between Dilution Rate and Substrate Concentration

When dilution rate in Example 1 was changed from 0.1/h to 0.5/h, almost all of the substrate was consumed, and productivity increased with an increase in dilution rate. On the other hand, when the concentration of glucose was sequentially increased to 100 g/L, 150 g/L and 200 g/L, glucose was almost consumed at low dilution rate up to a glucose concentration of 150 g/L. However, at high dilution rates such as 0.4/h and 0.5/h, glucose remained, and when the concentration of glucose was 200 g/L, glucose was not substantially consumed and thus was mostly discharged from the reactor.

That is, it can be seen that, when a first bioreactor, a second bioreactor, or if necessary, third or more bioreactors, are disposed, it is important to optimize the concentration and feed rate of substrate in each of the reactors.

### Example 5

### Treatment of Wastewater Using Single CSTR Bioreactor System

A bed, in which 35 cm³ of activated carbon was packed in a column having a diameter of 2.5 cm and a length of 10 cm, was disposed in a fermenter having a volume of 2.5 L (an effective volume of 1 L), thus constructing a single CSTR bioreactor system. Microorganisms collected from activated sludge of the Daejeon Sewage Treatment Plant were used in wastewater treatment, and influent water having a glucose concentration of 560 mg/l (on the basis of COD) was used. Also, in order to remove surplus microorganisms generated in the upflow packed bed, the packed bed was back-flushed with air using a disposable piston at intervals of 1-2 hours.

The results of wastewater treatment conducted at various residence times and oxygen concentrations are shown in Table 1 below. As shown in Table 1, when the residence time of microorganisms in the bioreactor was 4 hours, the steady-state was reached after approximately 20 hours, and thus a microbial treatment efficiency of more than 97% was shown. Also, when the residence time was 2 hours, the steady-state was reached after about 20 hours, and a microbial treatment efficiency of 80% was shown.

In order to prevent microorganism inhibition caused by a decrease in oxygen concentration and increase treatment efficiency, oxygen corresponding to two times the oxygen concentration of air was supplied, and thus a microbial treatment efficiency of more than 90% could be obtained.

TABLE 1

| | influent | Effluent | Treatment | Treatment |
|---|---|---|---|---|
| Residence | water | water | efficiency | capacity |
| time | (mg/L) | (mg/L) | (%) | (kg/m³ · d) |
| 4 h (air) | 560 | 17 | 97 | 3.25 |
| 2 h (air) | 560 | 112 | 80 | 6.17 |
| 2 h (high-purity oxygen) | 560 | 56 | 90 | 6.94 |

### Example 6

### Production of Alcohol Using Two-Stage CSTR Bioreactor System

In the same conditions as in Example 1, the bioreactor system was operated at a dilution rate of 0.5/h in the first bioreactor, and thus the alcohol concentration in the steady state became 42 g/L; the cell concentration, 87 g/L; and the productivity, 21.5 g/L. Then, 100 g/L of glucose serving as a substrate was additionally supplied into the second bioreactor and, as a result, there was little or no change in the production of alcohol, but the alcohol concentration in the steady state was 87 g/L. This suggests that the concentration of products is increased in the two-stage CSTR bioreactor system compared to the one-stage CSTR bioreactor system.

### Example 7

### Production of Lactic Acid Using Two-Stage CSTR Bioreactor System

In continuous lactic acid studies conducted using a two-stage hollow fiber cell recycle reactor system, the concentration of lactic acid in the first stage was 57 g/L, and the concentration of lactic acid in the second stage was 92 g/L (Kwon et al., Biotech. Bioeng., 73:25-34, 2001). As a result, it could be seen that the concentration of the product was increased in the two-stage CSTR bioreactor system compared to the one-stage CSTR bioreactor system.

### Example 8

### Production of Single Chain Antibody Using Two-Stage CSTR Bioreactor System

The CHO cell line 26*-320 of DUKX origin was cultured in protein free medium C-5467 (Sigma, St. Louis, Mo., USA) in a 2-L depth filter perfusion system (Lee, J. C. et al., Biotechnol. Prog., 21:134-139, 2005) at a perfusion rate of 6/d for 20 days. As a result, the concentration of a human platelet recombinant single chain antibody in the first bioreactor was 12.1 mg/L, and the concentration of the antibody in the second bioreactor was 23.9 mg/L. This suggests that the concentration of the product was increased in the two-stage CSTR bioreactor system compared to the one-stage CSTR bioreactor system.

### Example 9

### Production of Single Chain Antibody Using Multi-Stage CSTR Bioreactor System

The rCHO cell line CS*13-1.00 (chimeric antibody against S surface antigen) of DG44 origin was cultured in the two-stage reactor for 1800 hours in the same manner as in Example 8. When the depth filter was clogged because of the aggregation property of cells, the clogging phenomenon was solved using trypsin. The average concentration of the antibody in the two-stage culture system was 70 mg/L in the first stage and 100 mg/L (maximum: 160 mg/L, and minimum: 40 mg/L) in the second stage.

A model equation was made based on the above culture results, and the concentration value, which could be increased in the multi-stage CSTR bioreactor system, was tested through computer modeling.

The modeling was consistent with the batch and also consistent with the two-stage continuous process. The results of the computer modeling revealed that the average concentration of the antibody in each of the stages were 85 mg/L in the first stage, 114 mg/L in the second stage and 116 mg/L in third stage, suggesting that the difference in concentration between the second stage and the third stage was not great.

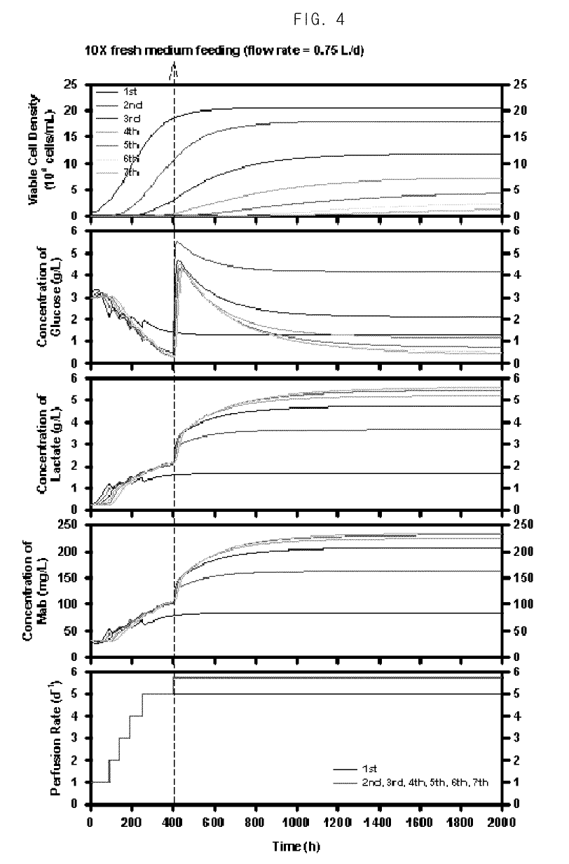

It was observed that a specific component in the medium was exhausted, and a test was carried out using a 10× concentrated medium. As a result, the average concentration of the antibody in the fourth stage was shown to be 200 mg/L. When the medium feeding rate was increased from 0.5 L/d to 0.7 L/d in order to increase the supply of nutrients, the average concentration of the antibody in the seventh stage was shown to be a maximum of 240 mg/L. However, an increase in the antibody concentration from the fourth stage was very insignificant. This concentration was the highest concentration which could be obtained when the same strain was cultured in a fed-batch manner (FIG. 4).

As a result, it could be confirmed that the multi-stage CSTR bioreactor system according to the present invention is capable of maintaining high productivity and, at the same time, maintaining the concentration of a product at a level almost equal to the concentration of a batch product.

### INDUSTRIAL APPLICABILITY

As described above, the present invention provides a multi-stage CSTR bioreactor system comprising a plurality of bioreactors, each equipped with a cell recycle unit for increasing the concentration of microorganisms in the bioreactors. According to the present invention, multi-stage culture is carried out in the bioreactors equipped with the cell recycle unit, and thus the production of alcohols, organic acids, antibiotics, recombinant proteins and the like, which are produced by anaerobic or aerobic microbial fermentation, can be increased, and in addition, a final product can be obtained at high concentration, thus improving the productivity and economic efficiency of a microbial process. Also, the multi-stage CSTR bioreactor system can be applied in wastewater treatment to effectively perform the removal of carbon sources and the removal of nutrients, such as nitrogen and phosphorus.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A single CSTR bioreactor system in which a cell recycle unit serving to block the discharge of cells from the bioreactor and, at the same time, discharge culture broth except for cells during a culture process, is equipped.

2. The single CSTR bioreactor system according to claim 1, wherein the cell recycle unit is selected from the group consisting of a packed bed, a cylindrical depth filter and a membrane cell recycle unit.

3. The single CSTR bioreactor system according to claim 2, wherein the packed bed is selected from the group consisting of a fully packed bed, a partially packed bed, a cylinder or pipe packed bed, a duct packed bed, a plate packed bed, and a multi-packed bed consisting of a plurality of said packed beds connected with each other.

4. The single CSTR bioreactor system according to claim 2, wherein the packed bed is packed with a packing material, selected from the group consisting of activated carbon, ceramic particles and polymer particles, to a porosity of 28-70%.

5. The single CSTR bioreactor system according to claim 1, wherein the cells are selected from the group consisting of bacteria, yeasts, molds, animal cells and plant cells.

6. A method for culturing microorganisms, the method comprises culturing said microorganisms in the single CSTR bioreactor system of claim 1.

7. The method for culturing microorganisms according to claim 6, wherein said microorganisms are selected from the group consisting of anaerobic, facultative and aerobic microorganisms.

8. The method for culturing microorganisms according to claim 6, wherein cell recycle units equipped in the bioreactor were rotated or backwashed to increase the efficiency of microorganism isolation.

9. A multi-stage CSTR reactor system in which the 2-7 single CSTR bioreactor systems of claim 1 are connected in series with each other.

10. A method for culturing microorganisms, the method comprises culturing said microorganisms in the multi-stage bioreactor system of claim 9.

11. The method for culturing microorganisms according to claim 10, wherein said microorganisms are selected from the group consisting of anaerobic, facultative and aerobic microorganisms.

12. The method for culturing microorganisms according to claim 10, wherein cell recycle units equipped in the bioreactor were rotated or backwashed to increase the efficiency of microorganism isolation.
